SEQUENCE LISTING (6 sequences)

SEQ ID NO: 1 - protein, 153 aa, Artificial Sequence
  synthetic construct
EFARSDVQLVESGGGLVQPGGSLRLTCAASGLIFGSYAMGWFRQAPGKAREFVAAISWSGGDTYADSVKGRFTISRDNAKNTVYLQMNSLEPEDTAVYSCAAVGSKYYISKDAKDYGYWGQGTQVTVSSAAAGGGGSGGGGSGSGLEHHHHHH

SEQ ID NO: 2 - protein, 13 aa, Artificial Sequence
  synthetic construct
AHIVMVDAYKPTK

SEQ ID NO: 3 - protein, 123 aa, Artificial Sequence
  synthetic construct
EVQLVQSGGGSVQRGGSLRLSCRHSSITVPDYTIGMFRRRPGKGGEEVSLISMHGGRSMYRGSVKGRFRISRDSVKNTVYLQMNNLKPEDTDIYYCGGTTFGLRRRPNEYDSMGQGSQVTVSS

SEQ ID NO: 4 - protein, 294 aa, Artificial Sequence
  synthetic construct
EVQLVQSGGGSVQAGGSLRLSCAHSSITVPDYTIGWFRRAPGKGGEEVSLISMHGGRSWYAGSVKGRFAISRDSVKNTVYLQMNNLKPEDTDIYYCGGTTFGLAAAPNEYDSWGQGSQVTVSSGGGGSGGGGSGSEVQLVQSGGGSVQAGGSLRLSCAHSSITVPDYTIGWFRRAPGKGGEEVSLISMHGGRSWYAGSVKGRFAISRDSVKNTVYLQMNNLKPEDTDIYYCGGTTFGLAAAPNEYDSWGQGSQVTVSSGGGGSGGGGSGSAHIVMVDAYKPTKAAALEHHHHHH

SEQ ID NO: 5 - protein, 148 aa, Artificial Sequence
  synthetic construct
EVQLVQSGGGSVQAGGSLRLSCAHSSITVPDYTIGWFRRAPGKGGEEVSLISMHGGRSWYAGSVKGRFAISRDSVKNTVYLQMNNLKPEDTDIYYCGGTTFGLAAAPNEYDSWGQGSQVTVSSAAAGGGGSGGGGSKKKALEHHHHHH

SEQ ID NO: 6 - protein, 139 aa, Artificial Sequence
  synthetic construct
EFARSVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGGGSGGGGSGSGLEHHHHHH

(19) United States
(12) Patent Application Publication
(10) Pub. No.: US 2020/0240982 A1
(43) Pub. Date: Jul. 30, 2020

(54) Use of Specific Covalent Bonding for Oriented Immobilization of Recombinant Antibody Fragments

(71) Applicant: The Government of the United States of America, as represented by the Secretary of the Navy, Arlington, VA (US)

(72) Inventors: George P. Anderson, Bowie, MD (US); Jinny Lin Liu, Ellicott City, MD (US); Ellen R. Goldman, Germantown, MD (US)

(21) Appl. No.: 16/746,119

(22) Filed: Jan. 17, 2020

Related U.S. Application Data
(60) Provisional application No. 62/797,472, filed on Jan. 28, 2019.

(51) Int. Cl. G01N 33/544; C07K 16/10; C07K 7/08

(52) U.S. Cl. CPC G01N 33/544; C07K 16/1081; C07K 2319/30; C07K 2317/569; C07K 7/08

(57) ABSTRACT

Expression of single-domain antibodies with a C-terminal binding partner (such as SpyTag) enables their orientation on surfaces, improving detection capability.

### CROSS-REFERENCE TO RELATED APPLICATIONS

This application claims the benefit of U.S. Provisional Application 62/797,472 filed on Jan. 28, 2019, the entirety of which is incorporated herein by reference.

### BACKGROUND

For detection applications, many immunoassays rely on recognition elements based on monoclonal or polyclonal antibodies (IgG) derived from mice, rabbits, goats, or sheep. Functional IgG are comprised of four polypeptide chains, two identical heavy (H) chains and two identical light (L) chains, linked by disulfide bonds. Each antibody has two antigen binding domains formed by the interaction of adjacent variable (V) domains from the H and L chains. The antigen binding surface is composed of six complementarity-determining regions (CDRs), three residing in each of the VII and VL protein domains. The interaction of these six CDR loops of varying sizes and sequences allows the formation of diversified antigen binding surfaces with the topologies to recognize a wide range of antigenic targets. Although sensitive and specific, conventional antibodies can be time-consuming and expensive to develop and are not easily molecularly engineered. FIG. 1 shows a schematic representation of IgG as well as the cloned binding derivative. Cloned derivatives of conventional IgG, comprising just the VII and VL domains to form a minimal antigen binding construct have been used as recognition elements for biosensor applications. These single chain antibodies (scFv) can be expressed in bacteria and modified by protein engineering to tailor the functionality and physico-chemical properties of the antibody fragments. An even simpler version is termed a single domain antibody (sdAb), whose origin is described below.

It was discovered that certain animals, such as camelids (i.e. camels, llamas) and sharks, possess a class of immunoglobulins consisting of heavy-chain homodimers where antigen binding is mediated through a single V domain (FIG. 1). These V domains, when recombinantly produced as single domain antibodies (sdAb), are the smallest known antigen binding fragments (12-15 kDa). Despite their small size, sdAb display a high level of specificity and affinity for their antigens and have been shown to have nanomolar affinities (K_{D}) for haptens and proteins. SdAb can re-fold to bind antigen after chemical or heat denaturation, enabling them to retain the ability to bind antigen after exposure to elevated temperatures. Several studies have found sdAb to be inherently thermostable, demonstrating antigen binding at elevated temperatures, which suggests they will be well suited for long-term field applications where refrigeration is often not possible. Recognition elements based on sdAb should offer the specificity of conventional antibodies with the potential for use and storage at elevated temperatures and the regeneration of sensor surfaces. SdAb provide stable, well-expressed binding elements with excellent affinity that can be tailored for specific applications through protein engineering.

While the recombinant antibody fragments, sdAb and scFv, offer several attractive properties relative to conventional antibodies, one limitation is that being small their covalent attachment to a surface in a random fashion can impair their binding function to a greater extent. To overcome this limitation, various methods have been used to orient the sdAb or scFv onto the capture surface. There have been a number of non-covalent methods examined to immobilize these antibody fragments including the incorporation of a biotinylation tag. In this case a biotin binding molecule is immobilized to the surface and the sdAb biotin fusion is then captured. This can work well but has limitations for many systems that utilize avidin-biotin interactions to generate the signal as its use for both parts of the sandwich assay leads to unacceptable background signals that reduces sensitivity. An alternative is to make a fusion of the sdAb with a biotin binding molecule, such as rhizavidin or strepcore avidin, in that case one needs to prepare a biotin surface and then allow the fusion sdAb-rhizavidin to bind down. Again this is not a suitable approach when avidin-biotin binding is used in later assay steps. Another limitation of any non-covalent immobilization is that even when the affinities of that interaction are quite high, in multiplexed situations such as one has with MagPlex microspheres (color coded microspheres that can be used in as much as a 50-plex assay) over extended storage time of the mixed microspheres some of the antibody could switch places, possibly leading to either false positives or negatives.

A further consideration in conducting assays is that complex matrices, such as plasma and serum, can dramatically reduce assay sensitivity. Thus, to achieve highly sensitive detection in complex matrices a highly effective assay is essential.

A need exists for techniques to improve limits of detection in assays using sdAb and other recombinant antibody fragments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustration showing the multi-domain nature of most antibodies and recombinant derivatives, but not sdAb. The heavy variable domains are shown in black, light domains in grey and the constant domains in white.

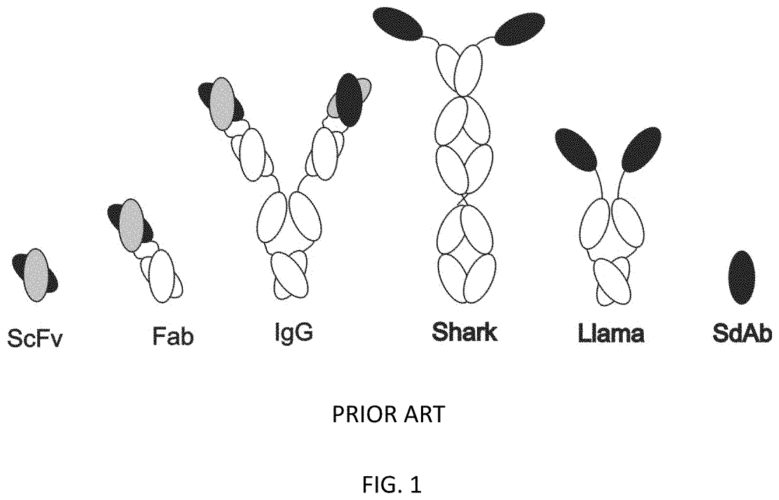

FIG. 2 shows results of MagPlex sandwich fluoroimmunoassays for the detection of Staphylococcal enterotoxin B (SEB) comparing different AcVe (A)-fusions and immobilization either directly or via use of SpyCatcher.

FIG. 3 shows results with various sdAb DD7 constructs which binds to Dengue NS1 protein as the capture molecule immobilized either covalently or via SpyCatcher for the detection of NS1 spiked into normal human serum (NHS).

FIGS. 4A and 4B show data with the tracers Bt-DD5-GS3K and Bt-DD1-GS3K, respectively, for the detection of Dengue NS1 spiked into serum by a sdAb DD7 construct covalently attached to microspheres directly and two sets of microspheres each coated with SpyCatcher and then a DD5-DD5-SpyTag construct (denoted here as XL-DD5-DD5).

### BRIEF SUMMARY

In one embodiment, an isolated single-domain antibody comprises a single domain antibody (sdAb) configured as a fusion protein comprising SpyTag (SEQ ID NO: 4) at a C-terminal of the sdAb.

In another embodiment, an isolated single-domain antibody comprises a genetically engineered fusion protein comprising (a) either SEQ ID NO: 3 or SEQ ID NO: 5; and (b) SpyTag (SEQ ID NO: 4) positioned at a C-terminal end of the fusion protein.

In a further embodiment, a method of detection, comprises providing a surface comprising SpyCatcher (SEQ ID NO: 6); providing a single domain antibody (sdAb) configured as a fusion protein comprising SpyTag (SEQ ID NO: 4) at a C-terminal of the sdAb; and contacting the surface with a sample suspected to contain an antigen recognized by the sdAb, wherein binding of the antigen to the sdAb results in detection of the antigen in the sample.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

Overview

The present inventors sought a technique to orient single domain antibodies (sdAb) to magnetic microspheres to improve the limits of detection achievable in immunoassays. A binding method involving partners interacting to form a covalent bond was found effective to orient single domain antibodies (sdAb) on a magnetic microsphere and improve their capacity to bind target antigen and improve limits of detection.

The examples described below use SpyTag and SpyCatcher, which are peptide-protein partners that lock together covalently, in order to orient sdAb. SpyCatcher was produced as a recombinant his-tagged protein followed by production of engineered SdAb-SpyTag-his tag fusion proteins. To prepare magnetic microspheres, the SpyCatcher was covalently immobilized using conventional EDC/sNHS chemistry. Incubation of the SpyCatcher-coated microspheres with the SdAb-SpyTag genetic fusion protein results in self-assembly to produce coated microspheres with the sdAb covalently attached in the desired orientation.

Aspects of this work were described in Anderson et al., “Oriented Immobilization of Single-Domain Antibodies Using SpyTag/SpyCatcher Yields Improved Limits of Detection,” Anal. Chem. 2019, 91, 15, 9424-9429 and its associated Supporting Information, incorporated herein by reference for the purposes of disclosing techniques for orientation of immobilized antibodies and their uses.

### EXAMPLES

To achieve the goal of preparing a MagPlex microsphere with oriented sdAb, a number of fusion proteins were prepared. First the surface of the microsphere was covalently coated with SpyCatcher, which is a small protein similar in size to the sdAb, which will spontaneously covalently bind with SpyTag. Different versions of the sdAb-SpyTag protein were also prepared. For the Staphylococcal enterotoxin B (SEB) assay, three fusion proteins were made using an anti-SEB sdAb, AcVe. There were a monomer (AcVe), dimer (AcVe-AcVe), and trimer (AcVe-AcVe-AcVe) all with the SpyTag genetically linked to the C-terminal. AcVe-SpyTag, AcVe-AcVe-SpyTag, and AcVe-AcVe-AcVe-SpyTag. Versions were also prepared that lacked the SpyTag. Then MagPlex microspheres were coated with these six proteins in the conventional covalent method using EDC and sNHS (1-ethyl-3-(3-dimethylaminopropyl)carbodiimide hydrochloride and N-hydroxysulfosuccinimide) chemistry, as well as the three SpyTag fusions were attached via the spontaneous reaction of SpyCatcher to SpyTag to microspheres that had first been coated with SpyCatcher using the EDC/sNHS chemistry. These 9 MagPlex bead sets were compared for their ability to detect SEB.

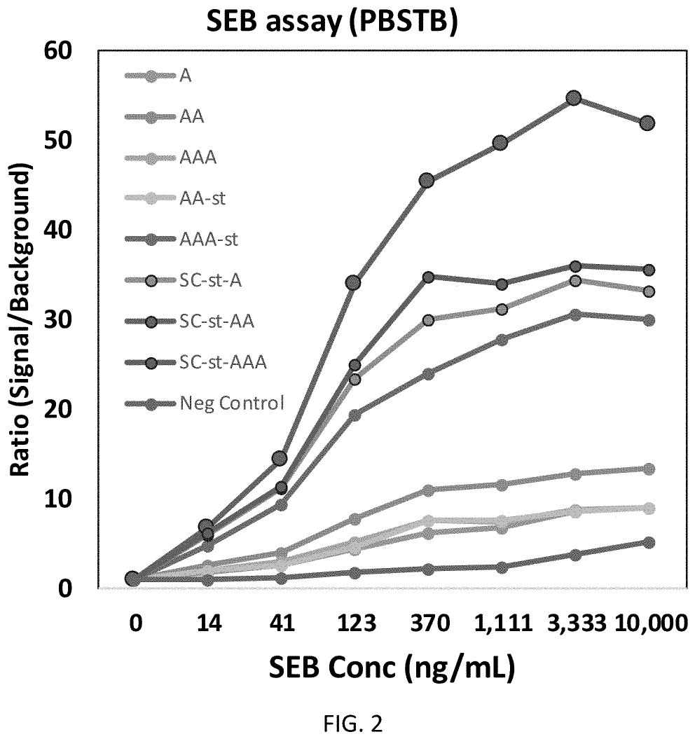

FIG. 2 shows results from MagPlex sandwich fluoroimmunoassays for the detection of SEB comparing different AcVe (A)-fusions. Shown are the A, AA, and AAA protein covalently attached in the standard way with EDC/sNHS chemistry. Very little difference in their ability to detect SEB was observed. With the AcVe-SpyTag (A-st), AA-st, and AAA-st fusions were covalently attached, a graduated improvement was seen going from monomer to dimer to trimer, for unclear reasons, but perhaps SpyTag provides some orienting effect during the immobilization. However, when those same sequences were attached via the immobilized SpyCatcher (SC), they were all much improved relative to the AcVe version lacking the SpyTag, and all were better than the chemically attached AAA-st. The best appeared to be the AA-st attached via SpyCatcher.

Using MagPlex microspheres first coated with SpyCatcher and then having the AcVe-SpyTag protein, monomer, dimer, or trimer, covalently bound provided a consistent effective surface, whereas the other assays produced either less signal or inconsistent signal levels. In order to confirm these results, a different assay was prepared using a sdAb-sdAb-SpyTag fusion protein immobilized onto the SpyCatcher coated MagPlex microsphere surface.

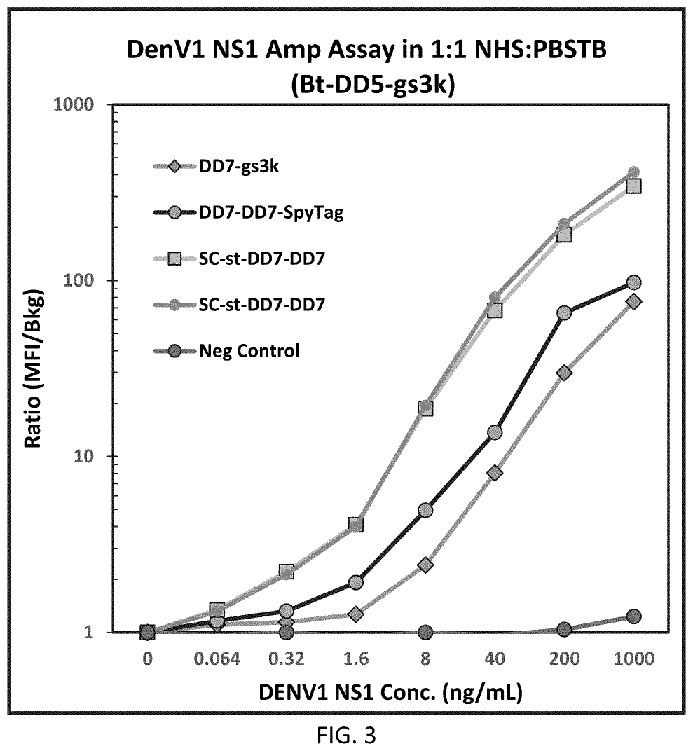

FIG. 3 shows results of an assay with the sdAb DD7 which binds to Dengue NS1 protein as the capture molecule. As with the SEB assay, it was prepared as a dimer with the SpyTag (DD7-DD7-SpyTag), when this construct was captured via SpyCatcher (SC) onto the microspheres surface it is shown as (SC-st-DD7-DD7) tested on two different sets of microspheres and compared to (DD7-DD7-SpyTag) covalently attached directly using EDC and sNHS or the monomer DD7 which included the C-terminal three lysine tail (DD7-gs3k) that partially orients the DD7 onto the surface using the EDC sNHS crosslinking

The result of the assay for the Dengue NS1 spiked into normal human serum (NHS) confirmed what was initially observed for the SEB assay, in that the use of a capture surface first coated with SpyCatcher in a random fashion, which is then used to capture the recombinant antibody (preferably a dimer i.e. sdAb-sdAb-SpyTag) provides a superior capture surface to alternative covalent methodologies. In the example shown in FIG. 3, the ability to detect NS1 improved by more than a factor of 5 over the same construct immobilized directly via EDC sNHS, and nearly 25-fold better than the DD7-gs3k construct that provided partial orientation. In each case detection was 0.32 ng/mL or better This huge improvement was most remarkable and now enables recombinant antibodies to achieve highly sensitive immunoassays

This technique was also used with antibodies to dengue virus (DENY), which exists as four antigenically distinct virus serotypes DENV-1 through DENV-4. The starting sdAb are described in Shriver-Lake, L. C., Liu, J. L., Zabetakis, D., Sugiharto, V. A., Lee, C., Defang, G. N., Wu, S. L., Anderson, G. P., Goldman, E. R. 2018 “Selection and Characterization of Anti-Dengue NS1 Single Domain Antibodies” Scientific Reports 8:18086. doi: 10.1038/s41598-018-35923-1 and associated Supplementary Information, incorporated herein by reference for disclosing a number of sdAb against DENY and techniques for producing additional such antibodies. A llama was immunized with a mixture of recombinant nonstructural protein 1 (NS1) antigen from the four DENV serotypes and a phage display immune library of single domain antibodies was constructed and to select sdAb with specificity and affinity for DENV NS1.

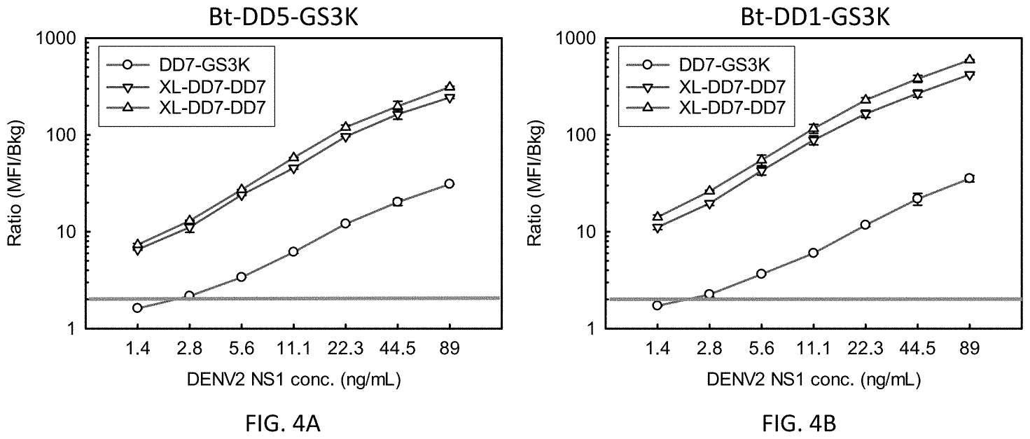

The multiplex nature of the MAGPIX instrument (Luminex Corp., Austin, Tex.) enabled testing of both old and improved sdAb immobilization protocols in parallel. FIGS. 4A and 4B show detection of NS1 from the DENV 2 serotype. The assay was performed in 50% normal human serum (NHS) and 50% LowCross buffer. The data is plotted as a ratio of signal (median fluorescent intensity; MFI) to background. The data show both the old (DD7-GS3K capture) and new formats (XL-DD7-DD7 oriented dimer capture, representing SpyCatcher coated microsphere binding the DD7-DD7-SpyTag). Sets employing the Bt-DDS-GS3K tracers are shown in FIG. 4A while data with a different tracer (Bt-DD1-GS3K) appears in FIG. 4B, demonstrating the improvement is independent of the tracer antibody being used. Measurements were done in duplicate; error bars represent the standard error. A ratio of above 2 was defined as positive (horizontal bar in the figures). These results demonstrate that the new immobilization protocol provides a substantial improvement in the signal to background ratio for the detection of DENV2 NS1 across the range of concentrations tested and clearly demonstrated an improved limit of detection (LOD) with 1.4 ng/mL being below the LOD by the old method while being easily detectable using the improved method.

Protein Sequences

The ACVE sdAb used in the examples has the sequence EFARSDVQLVESGGGLVQPGGSLRLTCAASGLIFGSYAMGWFRQAPGKA REFVAAISWSGGDTYADSVKGRFTISRDNAKNTVYLQMNSLEPEDTAVYS CAAVGSKYYISKDAKDYGYWGQGTQVTVSSAAAGGGGSGGGGSGSGLE HHHHHH (SEQ ID No: 1).

The Spy tag sequence is AHIVMVDAYKPTK (SEQ ID NO: 2).

The DD7 sdAb used here has the sequence EVQLVQSGGGSVQRGGSLRLSCRHSSITVPDYTIGMFRRRPGKGGEEVSLI SMHGGRSMYRGSVKGRFRISRDSVKNTVYLQMNNLKPEDTDIYYCGGT TFGLRRRPNEYDSMGQGSQVTVSS (SEQ ID NO: 3)

The construct XL-DD7-DD7, comprising a dimer of the DD7 sdAb and the orienting Spy tag sequence has the sequence: EVQLVQSGGGSVQAGGSLRLSCAHSSITVPDYTIGWFRRAPGKGGEEVSLI SMHGGRSWYAGSVKGRFAISRDSVKNTVYLQMNNLKPEDTDIYYCGGT TFGLAAAPNEYDSWGQGSQVTVSSGGGGSGGGGSGSEVQLVQSGGGSV QAGGSLRLSCAHSSITVPDYTIGWFRRAPGKGGEEVSLISMHGGRSWYA GSVKGRFAISRDSVKNTVYLQMNNLKPEDTDIYYCGGTTFGLAAAPNEY DSWGQGSQVTVSSGGGGSGGGGSGSAHIVMVDAYKPTKAAALEHHHH HH (SEQ ID NO: 4).

The construct DD7-gs3k had the sequence: EVQLVQSGGGSVQAGGSLRLSCAHSSITVPDYTIGWFRRAPGKGGEEVSLI SMHGGRSWYAGSVKGRFAISRDSVKNTVYLQMNNLKPEDTDIYYCGGT TFGLAAAPNEYDSWGQGSQVTVSSAAAGGGGSGGGGSKKKALEHHHH HH (SEQ ID NO: 5).

The SpyCatcher orienting protein which binds to SpyTag has the sequence: EFARSVDTLSGLSSEQGQSGDMTIEEDSATHIKFSKRDEDGKELAGATME LRDSSGKTISTWISDGQVKDFYLYPGKYTFVETAAPDGYEVATAITFTVN EQGQVTVNGKATKGDAHIGGGGSGGGGSGSGLEHHHHHH (SEQ ID NO: 6).

### Further Embodiments

It is expected the these techniques can be successfully used with a wide variety of sdAb beyond those employed in the examples, particularly because single domain antibodies are relatively simple compared to other antibodies.

One of ordinary skill in the art might modify the exemplary embodiments by adding or removing spacers (such as between the sdAb and SpyTag or a feature serving the same function), tags for affinity purification, and the like, and/or by employing sdAb multimers.

In some embodiments, multiple types of labeled microspheres might be mixed together for a multiplexed assay. In additional embodiments, each type of microsphere has a distinctive signature, for example a different color of dye.

In some embodiments, the capture surface demonstrated here, MagPlex microspheres, could be replaced by another type of solid support such as another type of microspheres or a planar surface. For example, the technique could be used on the surface of a microtiter plate, a sensor chip, etc. In such cases, binding of an antigen to immobilized can be detected using techniques known in the art.

A number of suitable binding partners exist which could yield the desired covalent bond formation. The SpyCatcher/SpyTag used in the examples might be replaced with another pair known in the art, for example SnoopCatcher/SnoopTag, SdyCatcher/SdyTag, and intein domains.

### Advantages

This technique is generic to recombinant binding elements of which the sdAb has been demonstrated. It should also be generic to many capture surfaces where the capture element is covalently immobilized in addition to the magnetic microsphere shown here. Thus this technique has the ability to improve any number of immunoassays where one can benefit by orientation of the capture molecule. The new feature is that it is possible now to first prepare the capture surfaces with a generic element, the SpyCatcher protein or similar functioning protein, and then by simple addition of the fusion protein in the examples shown, the sdAb-sdAb-SpyTag, one can prepare a capture surface with the capture molecule covalently attached.

The herein-described technique avoids problems posed by alternative approaches. There are a number of non-covalent method to prepare the capture surface in an oriented manner that do enhance the sensitivity. However, they have limitation in that the same method used to attach the molecule can cause high backgrounds if it also used to generate the signal being measured, i.e. on cannot use avidin-biotin to immobilize and then use a biotinylated recognition antibody followed by a avidin conjugated phycoerthryin to generate the signal. Also non-covalent methods can also be difficult to multiplex. Other methods to achieve covalent bonding with a desired orientation can be much more difficult to adapt to protein and may require a click-chemistry approach which is difficult and expensive to implement.

### Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

What is claimed is:

1. An isolated single-domain antibody comprising:
a single domain antibody (sdAb) configured as a fusion protein comprising SpyTag (SEQ ID NO: 4) at a C-terminal of the sdAb.

2. The antibody of claim 1, wherein the sdAb comprises SEQ ID NO: 3 or SEQ ID NO: 5.

3. The antibody of claim 1, having SEQ ID NO: 4.

4. The antibody of claim 1, in a state of being bound to a surface comprising SpyCatcher.

5. The antibody of claim 3, in a state of being bound to a surface comprising SpyCatcher.

6. An isolated single-domain antibody comprising:
a genetically engineered fusion protein comprising (a) either SEQ ID NO: 3 or SEQ ID NO: 5; and (b) SpyTag (SEQ ID NO: 4) positioned at a C-terminal end of the fusion protein.

7. A method of detection, comprising:
providing a surface comprising SpyCatcher (SEQ ID NO: 6);
providing a single domain antibody (sdAb) configured as a fusion protein comprising SpyTag (SEQ ID NO: 4) at a C-terminal of the sdAb; and
contacting the surface with a sample suspected to contain an antigen recognized by the sdAb, wherein binding of the antigen to the sdAb results in detection of the antigen in the sample.

8. The method of claim 7, wherein the fusion protein comprises SEQ ID NO: 3 or SEQ ID NO: 5.

9. The method of claim 7, wherein the fusion protein consists of SEQ ID NO: 4.
